(19) United States
(12) Patent Application Publication
(10) Pub. No.: US 2002/0188104 A1
(43) Pub. Date: Dec. 12, 2002

(54) Reaper protein

(75) Inventors: Sally A. Kornbluth, Durham, NC (US); Christopher Holley, Durham, NC (US)

Correspondence Address: Mary J. Wilson, NIXON & VANDERHYE P.C., 8th Floor, 1100 North Glebe Road, Arlington, VA, 22201-4714, US

(73) Assignee: DUKE UNIVERSITY

(21) Appl. No.: 09/924,102

(22) Filed: Aug. 8, 2001

Related U.S. Application Data
(60) Provisional application No. 60/223,699, filed on Aug. 8, 2000.

(51) Int. Cl. C07K 14/705; C07H 21/04; C12P 21/02; C12N 5/06

(52) U.S. Cl. 530/350; 536/23.5; 435/69.1; 435/325; 435/320.1

(57) ABSTRACT

The present invention relates to a human homolog of Drosophila melanogaster Reaper (hRpr) protein and to a nucleic acid sequence encoding same. The invention further relates to antibodies specific for hRpr and antisense molecules that bind nucleic acid sequences encoding hRpr. The invention also relates to methods of screening compounds for their ability to enhance or inhibit hRpr activity and to methods of using compounds so identified in therapeutic regimes.

This application claims priority from U.S. Provisional Patent Application Serial No. 60/223,699, filed Aug. 8, 2000, which is incorporated herein in its entirety by reference.

### TECHNICAL FIELD

The present invention relates to a human homolog of Drosophila melanogaster Reaper (Rpr) protein and to a nucleic acid sequence encoding same. The invention further relates to antibodies specific for human Reaper (hRpr) and antisense molecules that bind nucleic acid sequences encoding hRpr. The invention also relates to methods of screening compounds for their ability to enhance or inhibit hRpr activity and/or production and to methods of using compounds so identified in therapeutic regimes.

### BACKGROUND

Reaper, a 65 amino acid protein with no known enzymatic activity, is a central regulator of apoptotic cell death in the fruit fly, Drosophila melanogaster. In flies, synthesis of Reaper mRNA and protein is induced in response to multiple stimuli, including radiation and developmental signaling. Ectopic expression of fly Reaper in cells of lepidopteran and vertebrate origin (including human cells) results in apoptotic cell death. Conversely, a chromosomal deletion that includes Reaper abrogates virtually all programmed cell deaths in the fly.

In fly and human cells as well as Xenopus egg extracts, Drosophila Reaper protein has been shown to induce apoptosis through at least two protein-protein interactions: inhibition of IAPs (inhibitors of apoptosis), and through binding of the Scythe protein, which is believed to sequester mitochondrial cytochrome c-releasing factors. In both cases, Reaper expression promotes caspase activation and the apoptotic dismantling of cells. By these criteria, a bona fide human Reaper would be expected to interact with both IAPs and/or Scythe, and lead to caspase activation and morphological apoptosis. Further, as expression of fly Reaper immediately precedes apotosis, a putative human Reaper should be induced by apoptotic stimuli and be otherwise very tightly regulated.

The present invention provides a human protein product that fulfills the expected characteristics of a bona fide Reaper homolog.

### SUMMARY OF THE INVENTION

The present invention relates to a human homolog of the Drosophila melanogaster protein that is a central regulator of apoptotic cell death, Reaper, and to a nucleic acid sequence encoding same. The invention further relates to antibodies specific for human Reaper (hRpr) and antisense molecules that bind nucleic acid sequences encoding hRpr. The invention also relates to methods of screening compounds for their ability to enhance or inhibit hRpr activity and/or production and to methods of using compounds so identified in therapeutic regimes.

Objects and advantages of the present invention will be clear from the description that follows.

### BRIEF DESCRIPTION OF THE DRAWINGS

FIG. 1 Identification of hRpr. ClustalW amino acid sequence alignment of Drosophila Rpr (SEQ ID NO:1) with hRpr (SEQ ID NO:2). Initial database searching identified the Rpr-homologous region corresponding to amino acids 21-59 of hRpr. Arrows indicate predicted regions of α-helix formation as calculated by the PSIPRED and SAM-T99 algorithms. The Drosophila sequence is available from GenBank/EMBL/DDBL under accesssion number L31631. GenBank accesssion number AC005606 includes the hRpr encoding sequence.

FIGS. 2A-2D hRpr interacts with Scythe and is encoded by a radiation-inducible bicistronic mRNA.

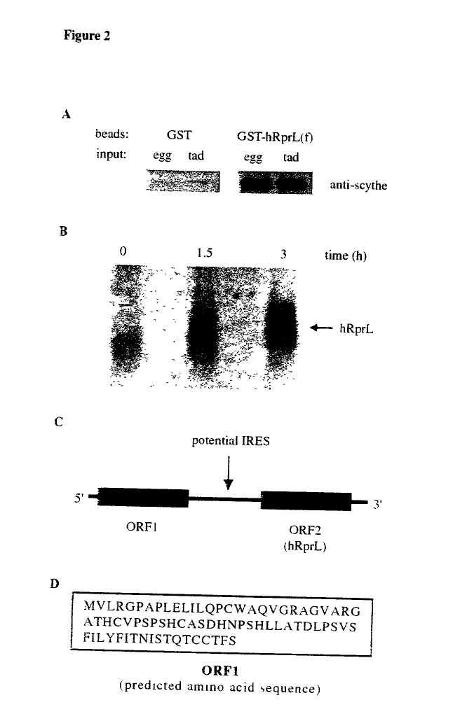

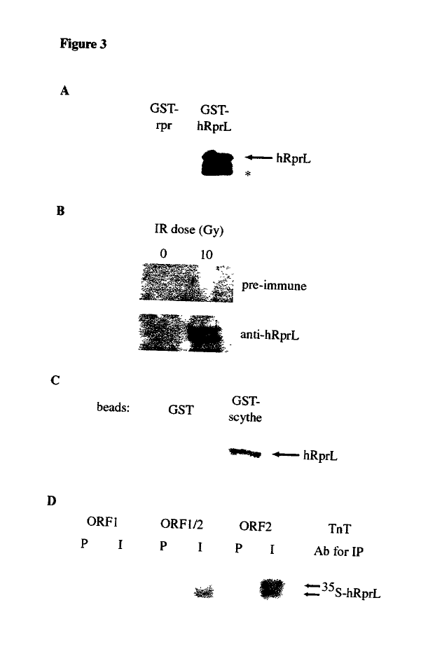

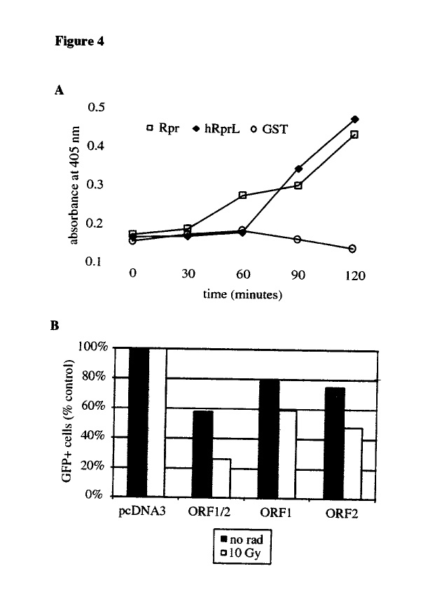

(FIG. 2A) The partial sequence of hRpr [hRpr(f)] identified initially by database searching was cloned in-frame with GST. Recombinant GST and GST-hRpr(f) proteins immobilized on glutathione-Sepharose were incubated with Xenopus egg and tadpole extracts, and the resulting bead-bound material was analyzed by SDS-PAGE and immunoblotting with anti-Scythe antibody.

(FIG. 2B) ML-1 cells were irradiated with 1 Gy of IR. As shown, IR induced the hRpr message within 1.5 h.

(FIG. 2C) Cartoon illustrating the bicistronic nature of the hRpr cDNA, including the putative IRES.

(FIG. 2D) Conceptual translation of the ORF (ORF1) lying upstream of the hRpr coding sequence (SEQ ID NO:3).

FIGS. 3A-3D hRpr immune serum recognizes a 9 kD doublet in IP/immunoblot analyses.

(FIG. 3A) The hRpr immune serum recognized GST-hRpr specifically with no cross-reactivity to a similarly sized GST-Rpr fusion. The * indicates breakdown products of the recombinant GST-hRpr.

(FIG. 3B) ML-1 cells were irradiated with 10 Gy and cell lysates were made at the indicated time points for IP and immunoblotting using hRpr immune and preimmune sera. The hRpr immune serum specifically recognizes a protein doublet of approximately 9 kD, the predicted molecular mass of the protein encoded by the hRpr ORF.

(FIG. 3C) ML-1 cells were irradiated with 100 Gy, and a cell lysate was made after 2.5 h. Bead-bound GST and GST-Scythe were then incubated with this extract, and the presence of hRpr in the bead-bound material was detected by immunoblotting.

(FIG. 3D) ³⁵S-labeled products from in vitro transcription and translation reactions programmed with the full-length hRpr cDNA (ORF1/2), the hRpr ORF alone (ORF2), or ORF1 alone were immunoprecipitated with preimmune serum (P) or hRpr immune serum (I) and analyzed by autoradiography. Both constructs containing the hRpr ORF produced an immunoprecipitable hRpr polypeptide doublet, whereas the construct containing ORF1 alone produced a ³⁵S-labeled product which was not precipitable with hRpr immune serum.

FIGS. 4A and 4B hRpr leads to caspase activation and cell death.

(FIG. 4A) Xenopus egg extracts were incubated with either GST or fusion proteins made with the complete hRpr ORF or fly Rpr. These extracts were then analyzed for caspase activation. The GST-hRpr fusion resulted in caspase activation with similar kinetics to Drosophila Rpr.

(FIG. 4B) HeLa cells were transiently transfected with equal amounts of GFP and the constructs indicated. After 12 h, the cells were exposed to 10 Gy of IR as indicated. After 8 more hours, the cells were washed, fixed, and counted. The percent of GFP-positive nuclei in each experiment are reported relative to controls.

### DETAILED DESCRIPTION OF THE INVENTION

The present invention relates generally to a human homolog of the Drosophila Reaper protein (hRpr).

In one embodiment, the present invention provides an isolated hRpr polypeptide. Preferably, the polypeptide comprises the amino acid sequence of SEQ ID NO:2.

In another embodiment, the present invention provides an isolated polynucleotide (DNA or RNA) that encodes a hRpr polypeptide. Preferably, the polynucleotide is a DNA molecule, more preferably, an isolated polynucleotide comprising the nucleotide sequence of SEQ ID NO:4.

The present invention also provides a vector comprising a polynucleotide that encodes a hRpr polypeptide. In a preferred embodiment, the vector is an expression vector in which the polynucleotide is operatively linked to a promoter.

The invention also relates to a recombinant cell transfected with a polynucleotide that encodes a hRpr polypeptide. Preferably, the cell is transfected with the above-described expression vector.

In yet another embodiment, the present invention relates to a method of producing a hRpr polypeptide by culturing the above-described preferred recombinant cell under conditions suitable for expression of the polypeptide.

The invention also relates to an antibody specific for a hRpr polypeptide. The antibody can be either monoclonal or polyclonal.

The invention also relates to a kit comprising, for example, a polypeptide, polynucleotide or antibody as described above, advantageously disposed within a container means. Such kits can be used to detect the presence of, for example, a hRpr polypeptide or polynucleotide of the invention.

The present invention further relates to a method of screening a test compound for its ability to interact with (e.g., bind to) a hRpr polypeptide encoding sequence.

As used herein, the term “isolated” means separated “by the hand of man” from its natural state; i.e., that, if it occurs in nature, it has been changed or removed from its original environment, or both. For example, a naturally occurring polynucleotide or a polypeptide naturally present in a living organism in its natural state is not “isolated,” but the same polynucleotide or polypeptide separated from coexisting materials of its natural state is “isolated”, as the term is employed herein. As part of or following isolation, such polynucleotides can be joined to other polynucleotides, such as DNAs, for mutagenesis, to form fusion proteins, and for propagation or expression in a host, for example. The isolated polynucleotides, alone or joined to other polynucleotides such as vectors, can be introduced into host cells, in culture or in whole organisms. Introduced into host cells in culture or in whole organisms, such DNAs still are “isolated”, as the term is used herein, because they would not be in their naturally occurring form or environment. Similarly, the polynucleotides and polypeptides can occur in a composition, such as media formulations, solutions for introduction of polynucleotides or polypeptides, for example, into cells, compositions or solutions for chemical or enzymatic reactions, for example, which are not naturally occurring compositions, and, therein remain “isolated”. Consistent with the foregoing, “isolated” does not encompass a human being comprising a polynucleotide or polypeptide of the invention.

Polypeptides/Proteins

As indicated above, the present invention relates, in one embodiment, to an isolated hRpr polypeptide, advantageously, the polypeptide having the sequence shown in SEQ ID NO:2. In addition to the SEQ ID NO:2 sequence, hRpr polypeptides include naturally occurring allelic variants of the SEQ ID NO:2 sequence. Non-naturally occurring variants of the SEQ ID NO:2 sequence are also within the scope of the invention and include polypeptides in which certain amino acids are substituted for other amino acids in the SEQ ID NO:2 sequence without appreciable loss of hRpr function. Table 1 includes exemplary substitutions.

TABLE 1

| Original Residue | Exemplary Substitutions |
|---|---|
| Ala | Gly; Ser |
| Arg | Lys |
| Asn | Gln; His |
| Asp | Glu |
| Cys | Ser |
| Gln | Asn |
| Glu | Asp |
| Gly | Ala |
| His | Asn; Gln |
| Ile | Leu; Val |
| Leu | Ile; Val |
| Lys | Arg |
| Met | Met; Leu; Tyr |
| Ser | Thr |
| Thr | Ser |
| Trp | Tyr |
| Tyr | Trp; Phe |
| Val | Ile; Leu |

The invention also includes polypeptides that are homologous to the polypeptide of SEQ ID NO:2. Such homologous polypeptides can retain at least one function of the hRpr polypeptide, as defined above. Such polypeptides are, for example, 70% homologous to the SEQ ID NO:2 sequence, preferably 80% or 90% homologous and more preferably 95% homologous to the SEQ ID NO:2 sequence. The percent homology can be determined using the NCBI (National Center for Biotechnology Information) BLAST2 (Basic Local Alignment Search Tool2) algorithm (blastp). (The parameters are set to the following to ensure a standardized method: matrix=PAM30 (penalties: gap=9 and extend gap=1), dropoff=50, expect=10, word length=3, and filter off. Homology is based on two values reported by the BLAST2(blastp) algorithm for comparing two protein sequences: first, the algorithm must recognize that the protein in question is homologous to hRpr over at least 70% of hRpr's total length, which equates to 57 aa and is reported by the algorithm as the denominator of the fractional identities and positives; second, this greater than or equal to 57 amino acid region must additionally be at least 70% homologous to the hRpr sequence, as calculated by the algorithm and reported in the percentage positives. The following examples illustrate the point:

a. A hypothetical protein is compared to hRpr; output gives a positives fraction of 40/57 (70%). This protein is significantly homologous to hRpr by the stated criteria.

b. A hypothetical protein is compared to hRpr; output gives a positives fraction of 39/57 (68%). This protein is not significantly homologous to hRpr by the stated criteria (region of homology sufficient, but percent homology in the region insufficient).

c. A hypothetical protein is compared to hRpr; output gives a positives fraction of 40/56 (71%). This protein is not significantly homologous to hRpr by the stated criteria (region of homology not long enough).

d. Drosophila reaper, the only known homologue of hRpr, does not even qualify (positives 28/54, so insufficient by both criteria).

The present invention relates not only to the entirety of the hRpr protein, for example, the SEQ ID NO:2 sequence or variants thereof, but to portions thereof as well. The term “portions” relates to peptides and polypeptides of at least 10 or at least 16-18 amino acids in length, preferably, at least 30 or at least 50 amino acids and more preferably at least 75 amino acids. Examples of such portions include subsequences of the polypeptide of the invention that comprise amino acid sequences corresponding to the scythe binding domain (amino acids 21-59), to the putative IAP binding domain (amino acids 15-29) and to amino acids 36-55. These portions can be used, for example, as intermediates in the production of the full-length sequences or in the production of antibodies. The portions can retain at least one function of the hRpr polypeptide, as defined above.

The invention further includes fusion proteins that comprise an hRpr polypeptide (or variant thereof) fused, for example, to a protein such as glutathione-S-transferase (GST), green fluorescent protein (GFP) or intein. The invention also includes variants of the hRpr polypeptide that have been modified to include a short amino acid sequence or epitope (e.g., a “tag”) that is specifically recognized by an antibody, examples of which include c-myc, hemagglutanin (HA), FLAG and 6× His tags.

In certain embodiments, it is advantageous to employ a polypeptide of the present invention in combination with an appropriate label. A wide variety of appropriate labels are known in the art, including radioactive, fluorescent, enzymatic or other ligands, such as avidin/biotin, which are capable of giving a detectable signal.

A polypeptide of the present invention can be prepared by standard techniques well known to those skilled in the art. Such techniques include, but are not limited to, isolation and purification from tissues known to contain the polypeptide, and expression from DNA that encodes such a polypeptide using transformed cells. Variants of the SEQ ID NO:2 sequence can be produced using site specific mutagenesis (see Current Protocols in Molecular Biology, Ausbel, F M et al, eds. Wiley & Sons: USA (1996)).

Polynucleotides

The present invention relates, in a further embodiment, to an isolated polynucleotide (DNA or RNA) that encodes a hRpr polypeptide (or variant thereof or fusion protein comprising same). In a preferred embodiment, the polynucleotide of the present invention is a DNA molecule. Even more preferably, the polynucleotide encodes a polypeptide comprising the amino acid sequence of SEQ ID NO:2. Most preferably, the polynucleotide of the invention comprises the nucleotide sequence:

| | |
|---|---|
| 5′-ATGCTGCTGTCGACTCATTTATTTATTTACTTATTTATTTATTTTTTGAGT | (SEQ ID NO:4). |
| TACAGCCTGGGCGACAGAGCTAGACTCTGTCTCAGAAAAACAAAACAACAAC | |
| AAAAAGAAAAACAGATTCTAAGGCAAAGTGAAGTTTTATTCAGGAGTGAAAC | |
| TTTAAGGAAGACAGGGAAAAAGGGAAGGCGGTGGGGGGGGCAGGGGGGCAGG | |
| GGAGGGACAGCAGATACAGGCGGTATGTTCCTAAGCTGA-3′ | |

The invention includes not only a polynucleotide (DNA or RNA) encoding a full length hRpr sequence, but also a polynucleotide that encodes a portion of an hRpr (e.g., hRpr having the amino acid sequence of SEQ ID NO:2) suitable for use, for example, as a probe or primer. Such polynucleotides can be at least 15, preferably at least 30, more preferably at least 90, 150 or 225 nucleotides in length and can encode, for example, domains (regions) of hRpr such as the scythe binding domain (nucleotides 531-647) or the putative IAP binding domain (nucleotides 513-557). The invention also includes portions of the nucleotide sequence given in SEQ ID NO:4 not directly encoding the hRpr polypeptide, but which are otherwise involved with synthesis of the hRpr polypeptide, including but not limited to the putative internal ribosome entry site (IRES) (e.g., nucleotides 370-388) and other sequences that can regulate the synthesis of the hRpr polypeptide.

The present invention also relates to polynucleotides having a nucleic acid sequence substantially identical to the nucleic acid sequence of SEQ ID NO:4, or fragment thereof. A “substantially identical” sequence is one the complement of which hybridizes to the nucleic acid sequence of SEQ ID NO:4 in 6× saline/sodium citrate (SSC) containing 5× Denhardt's reagent, 0.5% SDS, 100 μg/ml denatured salmon sperm DNA, and which remains bound when subjected to washing at 68° C. to 42° C. with 2× SSC containing 0.5% SDS. (For details of reagent preparation, etc, see Sambrook et al, Molecular Cloning, A Laboratory Manual, 2nd Edition.)

The invention also includes polynucleotides that are homologous to the polynucleotide of SEQ ID NO:4. Homologous polynucleotides can encode proteins having at least one function of hRpr, as defined above. Such polynucleotides are advantageously 70% homologous to the SEQ ID NO:4 sequence, preferably 80% or 90% homologous and more preferably 95% homologous to the SEQ ID NO:4 sequence. The percent homology can be determined using the NCB1 BLAST2 algorithm (blastn). (The blastn program is set to default parameters and the two sequences are compared. Output of a homology region covering more than 70% of the hRPR-encoding region (170 bp) and being additionally 70% homologous over that region indicates significant homology, in a fashion similar to that described above in connection with polypeptides.)

The invention further relates to polynucleotides complementary to those described above.

As previously indicated, a polynucleotide of the invention can encode a polypeptide as described above. The invention, however, also includes polynucleotides that encode an antisense nucleic acid (see, e.g., Crooke, Progress in antisense technology: the end of the beginning, Methods Enzymol. 313:3-45 (2000) for methods of utilizing such antisense nucleic acids). In certain embodiments, it is advantageous to employ a polynucleotide of the present invention in combination with an appropriate label, for example, for detecting hybrid formation. A wide variety of appropriate labels are known in the art, including radioactive, fluorescent, enzymatic or other ligands, such as avidin/biotin, which are capable of giving a detectable signal.

A polynucleotide of the present invention can be prepared using standard techniques well-known to one of skill in the art and, if a DNA molecule, can be a cDNA molecule or a DNA molecule prepared from a genomic DNA library.

Vectors

The present invention further comprises a vector comprising a polynucleotide as described above. The vector can be any selected vector, including but not limited to a viral vector, a plasmid or an artificial chromosome, such as a yeast or bacterial artificial chromosome.

Viral vectors suitable for use in the invention include retroviral vectors, adenoviral vectors and adeno-associated viral vectors (see also Robbins et al, Pharmacol. Ther. 80(1):35-42 (1998)). Replication-defective retroviral shuttle vectors and complementing retroviral producing cell lines are well-known in the art (for an overview of the relevant technology see Current Protocols in Molecular Biology, Ausbel F M et al, eds. Wiley & Sons: USA (1996)). One specific example of this technology is an expression system that combines the retroviral replication-deficient vector pMSCV and the packaging cell line RetroPack pT67 (Clontech).

Plasmid vectors suitable for use in the invention include but are not limited to pcDNA3, pGEX-KG, pBluescript, pEBB and pET-42. Plasmid vectors include an origin of replication and be selected based upon the type of cell in which the plasmid is to be maintained. For example, plasmids to be used for transfection of mammalian cells can contain genes that confer two different types of drug resistance. These plasmids can first be propagated in bacteria by selection for, for example, ampicillin resistance. Then, mammalian cells can be isolated by selection for, for example, neomycin resistance.

Vectors of the invention can contain one or —more suitable selection markers. For example, suitable selection markers can include antibiotic resistance (e.g., chloramphenicol resistance, tetracycline resistance, ampicillin resistance, kanamycin resistance), auxotrophic markers and cell-surface markers. Additional vector components are known in the art, are suitable for use in the present vectors and include, for example, processing sites such as a polyadenylation signal, ribosome binding sites, RNA splice sites, and transcriptional termination sequences.

A polynucleotide of the invention can be present in the vector operably linked to a promoter. The promoter can be a prokaryotic promoter or a eukaryotic promoter, such as T7, Lac, cytomegalovirus (CMV) and human EF-1α promoters.

The IRES element of the invention can be used (in a manner comparable to Clontech's pIRES vectors (which contain an IRES element from the encephalomyocarditis virus (ECMV)) to effect the linked expression of two distinct genes from a single transcriptional promoter.

Host Cells

The present invention further relates to host cells comprising a polynucleotide as described above, preferably, present in an above-described vector. Suitable host cells include procaryotic cells, such as bacteria (e.g., E. coli), lower eucaryotic cells, such as yeast (e.g., S. cerevisiae), and higher eucaryotic cells such as mammalian cells (e.g., human cells) and insect cells. Preferred host cells include but are not limited to the HeLa, MCF7, ML1, SF9, 293T and BHK21 cell lines, as well as various neuronally derived cell lines.

A polynucleotide of the invention can be introduced into a host cell as described above using any of a variety of art-recognized techniques. Means of transforming or transfecting host cells include calcium phosphate- or DEAE-dextran-mediated transfection, protoplast fusion, electroporation, liposome-mediated transfection, direct microinjection and viral infection (for overview see Current Protocols in Molecular Biology, Ausbel, F M et al, eds. Wiley & Sons: USA (1996)).

Host cells transformed/transfected with a polynucleotide encoding a polypeptide as described above can be used to effect production of that polypeptide. Typically, the host cells are cultured in a culture medium and under conditions such that the encoding polynucleotide is expressed and the polypeptide thereby produced. Optimum medium and culture conditions can vary with the host cell used and can be readily established by one skilled in the art. Once produced, the polypeptide can be isolated and purified as desired using standard techniques.

Antibodies

The invention additionally relates to antibodies specific for polypeptides (e.g., hRpr or variant thereof or portion thereof) described above. The antibodies can be monoclonal or polyclonal. The antibodies can be chimeric antibodies or humanized antibodies. The antibodies can be produced using art-recognized techniques such as those described in Harlow and Lane, Antibodies: A Laboratory Manual (Cold Spring Harbor Laboratories (1988)). In addition to whole antibodies, the invention also includes antibody fragments including Fab fragments. In a preferred embodiment, the antibody of the invention is specific for the polypeptide KEKQILRQSEVLFRSETLRK (SEQ ID NO:5).

Antibodies of the invention can be unlabeled or can bear a detectable label, e.g., a radiolabel or a fluorescent label. Other labels include gold (for electron microscopy) and biotin, as well as enzymatic labels, like horseradish peroxidase (HRP), that allow detection via a chemical reaction with a calorimetric or light-emitting substrate. The antibodies can be used in detection or purification protocols designed to detect or purify the polypeptide to which the antibody is directed. Where appropriate to facilitate use, the antibody of the invention can be bound to a solid support, for example, to a plastic or glass plate or bead, a column or a filter or membrane.

Kits

Any of the above-described polypeptides, polynucleotides, vectors or antibodies can be provided in a kit. Such a kit can comprise one or more of the polypeptides, polynucleotides, vectors or antibodies of the invention, labeled or unlabeled, disposed in one or more container means. The kit can include, for example, ancillary reagents (e.g., buffers) suitable, for example, for using the polynucleotides, polypeptides or antibodies in detection protocols (e.g., for detecting the presence of hRpr or hRpr encoding sequences in a sample).

Screening Assays

The present invention additionally relates to a method of screening a test compound for its ability to modulate the function of an hRpr polypeptide of the invention.

In one embodiment, the method comprises providing a polypeptide comprising an hRpr and determining the ability of a test compound to interact therewith. Such a method can be used to identify ligands for hRpr and thereby to determine the suitability of a test compound for potential use as an agonist or antagonist of hRpr. Screening assays of this embodiment generally involve first determining the ability of a test compound to bind hRpr. A compound that binds can then be tested for its ability to affect the activity of the hRpr. By way of example, an hRpr polypeptide of the invention can be coupled to a solid support, e.g., to plastic beads or plates, using well known coupling agents. Test compounds (which can bear a detectable label) can then be contacted with the immobilized polypeptide and the interaction between the test compound and the polypeptide monitored. Alternatively, competitive assays can be used wherein a known hRpr ligand (e.g., BAT3 (human scythe or HLA-B associated transcript 3)—GenBank accession number 179351), preferably bearing a detectable label, is used and the ability of a test compound to compete therewith for binding to hRpr is determined.

In addition to the above-described binding assays, function-based assays can also be used to screen test compounds for potential use as hRpr agonists or antagonists. Compounds that enhance or inhibit hRpr-induced apoptosis can be screened for based on the observation that hRpr, like its Drosophila counterpart, can act in conjunction with cytosolic factors to trigger cytochrome c release from the mitochondria (Evans et al, EMBO J. 16:7372-7381 (1997)). For example, mitochondria purified from Xenopus egg extracts can be aliquoted into 96-well plates. Isolated cytosol (e.g., prepared in parallel from Xenopus eggs) or cytosol immunodepleted of the hRpr-interacting protein, Scythe, can be added to the wells. This array can then be used to screen test compounds for their ability to trigger cytochrome c release from mitochondria in the presence of Scythe (i.e., Reaper-mimetics) or to screen test compounds for their ability to enhance or inhibit cytochrome c release upon addition of hRpr (e.g., recombinant). Cytochrome c release can be measured via an ELISA using anti-cytochrome c antibodies or fluorometrically using mitochondria pre-loaded with GFP-cytochrome c (Goldstein et al, Nature Cell Biol. 2:156 (2000)).

Compounds (candidate drugs) identified using screening methodologies such as those described above can be optimized using data obtained by subjecting hRpr to analytical techniques such as NMR and X-ray crystallography. These types of analyses can also be used in de novo drug design. In either case, the teachings of Hubbard (Curr. Opin. Biotechnol. 8(6):696-700 (1997)) are relevant.

Diagnostic and Therapeutic Applications

Potential diagnostic applications made possible by the identification of hRpr include, but are not limited to, assays to determine the genetic status of hRpr in an individual or tissue/blood/cell sample, and assays to determine the expression level of hRpr in a tissue, blood, or cell sample. For example, mutations that inactivate hRpr may be predictive of the resistance of tumors to radiation therapy. In that case, the genetic status of hRpr in a patient's tumor can be determined prior to radiation therapy. If hRpr is found to be mutated, an alternate therapy may be indicated.

Potential therapeutic applications made possible by the identification of hRpr include, but are not limited to, the use of hRpr mimics or enhancers or the use of compounds that inactivate hRpr, inhibit hRpr expression or otherwise prevent hRpr function. hRpr mimics or enhancers can act by-mimicking the role of hRpr in apoptosis or by enhancing the activity of endogenous hRpr. For example, an hRpr enhancer (e.g., a small molecule (or drug)) can be delivered systemically to a cancer patient. Since most cells do not express hRpr, an enhancer so delivered would have virtually nothing with which to interact. However, cells at a tumor site that have been therapeutically irradiated will be induced to express hRpr. An enhancer can, under these circumstances, interact with the hRpr that is expressed specifically in the irradiated (tumor) tissue resulting in even more apoptosis than would result from radiation alone. Lower doses of radiation, with more precise targeting of the tumor tissue and greater sparing of normal tissue, can be expected. In another example, a compound can be administered that acts to inactivate hRpr. In diseases that result from inappropriate apoptosis due to the overexpression of hRpr (such as various neurodegenerative and autoimmune diseases), treatment can be aided by administration of a compound that blocks the activation of apoptosis by hRpr.

hRpr enhancers and an inactivators can be administered in a variety of ways, including but not limited to oral, intravenous, intramuscular, and topical administration. One skilled in the art can readily establish optimum administration regimens.

Certain aspects of the present invention are described in greater detail in the non-limiting Example that follows.

### EXAMPLE 1

Experimental Details

Cloning

The initial hRpr fragment (f) was identified in the non-redundant database with the NCBI Advanced BLAST algorithm after the defaults were changed to PAM 30 scoring matrix, filter off, expect=100. Full-length Rpr protein was the query (Accession Q24475). HRpr(f) was amplified from human genomic DNA by nested PCR using the primers: [Round 1] Fwd1 (5′-TCCCTCTGTGCTAACTCCTTTGG) (SEQ ID NO:6) and Rev1 (5′-CCCCAAGCTTACCTTCCCTTTTTCCCTGTCTTC) (SEQ ID NO:7), and [Round 2] Fwd0 (5′-GCGCATCCATGGGCGACAGAGCTAGACTC) SEQ ID NO:8) and Rev1. This fragment was cloned into pGEX-KG to make the glutathione-S-transferase (GST)-hRpr(f) construct. To obtain the complete coding sequence, a cDNA library for rapid amplification of cDNA ends (RACE) was synthesized using a Marathon cDNA Amplification Kit (Clontech). 5′-RACE utilized the Expand High Fidelity PCR System (Roche) in conjunction with Marathon adapter primer AP1 and hRpr primer Rev1. The primers Fwd8 (5′-CCACCCTGTACTTCAGATAATAAGCAACCC) (SEQ ID NO:9) and Rev5L (5′-TGGGCCCTCTGGGAAGGCTGTG) (SEQ ID NO:10) were used together with the GC-Rich PCR system (Roche) to amplify the full cDNA, which was cloned into pcDNA3 for the ORF1/2 construct. The hRpr ORF alone was then PCR-amplified with Fwd11 (5′-GGAATTCTGATGCTGCTGTCGACTCATTTA) (SEQ ID NO:11) and Rev5L, and cloned in frame with GST in pGEX-KG to form GST-hRpr. The ORF1 and ORF2 constructs each contain a single open reading frame whose initiation Met is in the context of a Kozak consensus (GCC ATG G). These were PCR amplified and cloned into pcDNA3 using the primers [K-ORF1] Fwd13 (5′

| | |
|---|---|
| [K-ORF1] Fwd13 | |
| (5′-CCGAAGCTTGCCATGGTGCTGCGTGGCCCAGCC) | (SEQ ID NO:12) and |
| Rev8 | |
| (5′-TGAATTCTTAGGAGAAAGTGCAGCATGTCTGGGTG) | (SEQ ID NO:13), |
| [K-ORF2] Fwd12 | |
| (5′-AGGAAGCTTGCCATGGTGCTGTCGACTCATTTATTT) | (SEQ ID NO:14) and |
| Rev5L. | |

RNA Isolation and Northern Blotting

Cells were irradiated using a Mark I cesium source (8.23 Gy/min). Total RNA was isolated from ML-1 cells using Trizol (Gibco). For Northerns, 30 μg of total RNA/lane was separated by electrophoresis and transferred to nitrocellulose. The membrane was baked, prehybridized (5×SSC, 50 mM NaPO₄ (pH 7.4), 1× Denhardt's, 100 μg/mL fish sperm), and then hybridized at 65° C. with ³²P-labeled probe synthesized by PCR (primers Fwd0 and Rev1). Afterward, the blot was washed in 0.2× SSC/0.1% SDS at 65° C. and autoradiographed.

For cDNA synthesis, ML-1 cells were irradiated with 1.4 Gy, and total RNA was isolated after 3 h. PolyA+ mRNA was isolated using PolyA Quik (Stratagene)

GST-Fusion Protein Purification and Binding Assays

GST-proteins were expressed and purified in E. coli, and interphase Xenopus egg extracts were prepared in egg lysis buffer (ELB) as described (Evans et al, EMBO J. 16:7372 (1997)). Tadpole extract was made by homogenization in 10 mM Hepes, 25% glycerol, 2 mM MgCl₂, 0.5 mM EDTA, 0.5 mM DTT, and 5 μg/mL each of aprotinin and leupeptin. For binding assays, 50 μl bead-bound GST-fusion protein was incubated with 100 μl of extract plus 100 μl of ELB. Bead-bound material was washed 3× with ELB and separated by SDS-PAGE on a 12% gel prior to immunoblotting. Extracts supplemented with recombinant proteins were assayed for caspase activity by monitoring cleavage of Ac-DEVD-pNA (Thress et al, EMBO J. 17:6135 (1998)).

Antibody Production, Immunoprecipitation (IP), and Immunoblotting

Rabbits were immunized with a peptide derived from hRpr (KEKQILRQSEVLFRSETLRK) (SEQ ID NO:5). For immunoprecipitation (IP), ML-1 cells were lysed in IP buffer (50 mM Hepes pH 7.5, 150 mM NaCl, 1 mM EDTA, 1% NP40) containing Complete Protease Inhibitor (Roche). For IPs, in vitro translation reactions were diluted with IP buffer, incubated with 5 μL of serum on ice, and then incubated with Protein A-Sepharose. Bead-bound material was washed 3× in IP buffer and separated on 17.5% gels. Immunoblots of proteins transferred to PVDF (positively charged nylon membrane—IMMOBILON (Millipore)) were blocked with 5% dry milk in PBS/0.1% Tween-20, and probed with either hRpr immune serum or anti-Scythe antibody at 1:1000 in PBS/0.1% Tween-20 with 1% BSA. Blots were developed by chemiluminescence after incubation with Protein A-HRP.

In vitro transcription/translation reactions were performed using the TnT Coupled Reticulocyte Lysate System (Promega), and ³⁵S-Met/Cys translabel (ICN). Immunoprecipitated products were separated on 17.5% SDS-PAGE gels, and visualized by fluorography.

Cell Culture and Transfections

ML-1 cells were maintained in RPMI 1640 with 10% FBS. HeLa cells were maintained in MEM with 10% FBS, non-essential amino acids, and sodium pyruvate. For cell death assays, HeLa cells were plated on glass coverslips in 6-well dishes. Cells were transfected with 0.5 μg each of green fluorescent protein (GFP) and the indicated construct, using Fugene 6 (Roche). After 12 h, cells were irradiated as indicated. Eight hours later, the cells on coverslips were washed 1× in PBS, fixed for 10 minutes in fresh 4% paraformaldehyde in PBS, washed 2× more in PBS, and air dried. Coverslips were mounted on slides with Vectashield (Vector Labs) and examined by phase contrast and fluorescence microscopy. GFP-positive cells was scored by counting all the cells in a random field by phase contrast and then switching to fluorescence to count the GFP-positive cells in that field.

Results

Identification of a Putative Rpr Homolog

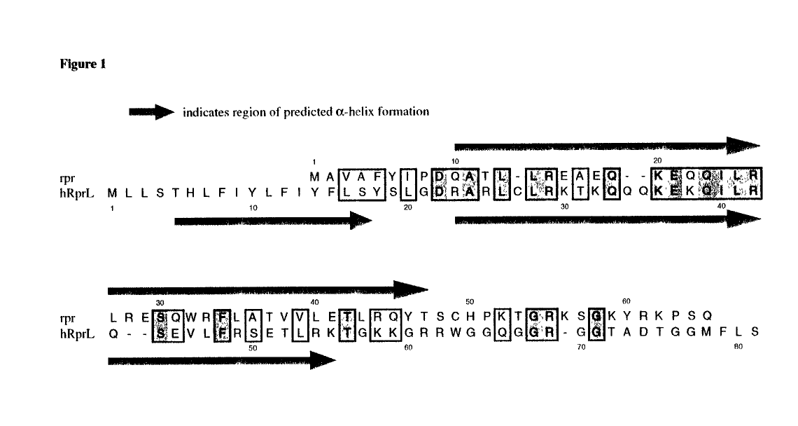

The search for vertebrate Rpr-like proteins began with a search of public databases for sequences similar to fly Rpr. Conventional searches of genomic and expressed sequence tag (EST) databases proved unsuccessful, but an Advanced BLAST search identified DNA from human chromosome 16, the theoretical translation of which had 41% amino acid identity and 51% similarity to a large portion of Drosophila Rpr (FIG. 1). This sequence lacked an apparent start codon, but was preceded by a potential splice acceptor.

To determine whether the conceptual polypeptide encoded by this sequence had biochemical properties similar to fly Rpr, the sequence was amplified by PCR from human genomic DNA and subcloned into a fusion vector for expression as a GST-hRpr fragment (GST-hRpr(f)). When this fusion protein was produced in bacteria and incubated in Xenopus egg or tadpole extracts, it successfully bound Scythe protein in both extracts (FIG. 2A), demonstrating that hRpr shares this characteristic property with Rpr.

Isolation of a Bicistronic hRpr-Encoding cDNA

The sequence homology of the hRpr fragment to fly Rpr and the ability of hRpr to bind Scythe prompted an attempt at cloning a cDNA corresponding to the genomic hRpr sequence. As indicated above, a clone corresponding to hRpr was not detected in any EST database, nor was it possible to identify a related clone by PCR and hybridization screens of several libraries. Since fly Rpr is transcriptionally induced by p53 in response to ionizing radiation (IR) (Brodsky et al, Cell 101:103 (2000)), an investigation was made of the induction of a hRpr transcript following γ-irradiation of ML-1 cells (human myeloid leukemia cells with wild-type p53). Northern blots made from irradiated ML-1 cells and probed with DNA encoding the hRpr fragment revealed a message of 0.8 kb that was induced within 1.5 h in response to 1 Gray (Gy) of IR (FIG. 2B). Given this, mRNA from irradiated ML-1 cells was used to synthesize a cDNA library. The library was then used to obtain a clone encoding hRpr using the RACE technique. Sequencing of the cDNA revealed that the presumptive splice acceptor was utilized to join two exons and produce a continuous ORF encoding a protein of 81 amino acids with a predicted molecular mass of 9.3 kD, slightly larger than fly Rpr (FIG. 1). The homology of hRpr to Rpr was corroborated by secondary structure analysis of the two proteins; strikingly, both Rpr and the hRpr proteins shared a large predicted α-helical domain, as calculated by two widely used secondary structure algorithms, PSIPRED and SAM-T99 (FIG. 1) (Jones, J. Mol. Biol. 292:195 (1999); Karplus et al, Bioinformatics 14:846 (1998)).

Sequence analysis of the largest cDNA clone, which corresponded in length to the 0.8 kb message seen on Northerns, revealed that the message actually encoded two non-overlapping ORFs. The Upstream ORF (ORF1) encoded a 75 amino acid peptide with no obvious similarity to previously characterized proteins, while hRpr was encoded by the second ORF (ORF2) (FIGS. 2C and 2D). The inter-ORF region contained a polypyrimidine tract that may serve as a site for internal ribosome entry. hRpr is synthesized in response to IR

Given the presence of an ORF upstream of the hRpr-encoding sequences, it was critical to show that hRpr was synthesized by apoptotic human cells. Therefore, an antibody was raised against a C-terminal peptide derived from hRpr. Immunoblotting of GST-hRpr and GST-Rpr proteins demonstrated specific reactivity of hRpr antibody against the human protein (FIG. 3A); this antibody was then used for IP and immunoblotting of lysates from irradiated and control ML-1 cells. Following 10 Gy of IR, the ML-1 cells synthesized a 9 kD doublet recognized specifically by hRpr immune serum (FIG. 3B). The induced hRpr bands were first detected at 2 h and peaked in abundance between 4 and 8 hr. Caspase activation followed hRpr production and was detectable by enzymatic assays 4 h after irradiation, while >30% of the cells had apoptotic morphology by 6 h. An anti-hRpr reactive protein was also precipitable by GST-Scythe, supporting the notion that the antibody was specific for hRpr-derived polypeptides (FIG. 3C).

To further validate translation from the hRpr ORF (ORF2) of the bicistronic mRNA, in vitro transcription/translation reactions were programmed with ORF1 alone, ORF2 (hRpr) alone, or the bicistronic ORF1/2 (the full-length cDNA) in the presence of ³⁵S Met/Cys. As shown in FIG. 3D, both ORF2 alone and the bicistronic ORF1/2, but not ORF1 alone, produced protein doublets immunoprecipitable by the anti-hRpr antibody, indicating that ORF2 (hRpr) can be translated in the context of the bicistronic message. Although bicistronic messages are rare in eukaryotes, a number of apoptotic regulators lie downstream of internal ribosome entry site (IRES) sequences (Coldwell et al, Oncogene 19:899 (2000); Henis-Korenblit et al, Mol. Cell. Biol. 20:496 (2000); Holcik et al, Nat. Cell Biol. 1:190 (1999)). While further analysis will be required to determine whether sequences upstream of ORF2 truly function as an IRES, the inter-ORF region does contain a polypyrimidine tract which is characteristic of IRES sequences (Holcik et al, Nat. Cell Biol. 1:190 (1999)). One interesting possibility is that radiation-inducible factors promote an IRES-driven increase in ORF2 translation.

Data shown in FIG. 3D demonstrate that the hRpr ORF2 alone can direct the synthesis of a 9 kD protein doublet, supporting the idea that the anti-hRpr immunoreactive doublet produced in irradiated ML-1 cells is encoded by the cDNA cloned. Possible explanations for doublet formation include post-translational modification or translational initiation at a non-methionine start codon.

Induction of Apoptosis by hRpr

To initially assess the apoptotic activity of the hRpr protein, GST-hRpr was added to cell-free extracts of Xenopus eggs. It was found that hRpr was an effective inducer of caspase activation (FIG. 4A). Moreover, hRpL-induced caspase activation, like that induced by GST-Rpr in egg extracts, was preceded by mitochondrial cytochrome c release.

To evaluate the apoptotic activity of hRpr in human cells, HeLa cells were co-transfected with a GFP construct and similar constructs encoding either ORF1 alone, ORF2 (hRpr) alone, or ORF1/2 (bicistronic). As shown in FIG. 4B, transfection with ORF2 (hRpr) alone led to a loss of GFP-positive cells, indicative of cell death. Transfection of ORF1 alone also resulted in a moderate loss of GFP-positive cells. However, transfection of the ORF1/2 construct (both ORFs in series) produced a more robust response, with a 45% reduction in GFP-positive cells compared to vector alone. This enhanced activity of hRpr in the presence of the ORF1 sequence did not appear to reflect enhanced production of hRpr, which was more abundant in the cells transfected with the hRpr ORF (ORF2) alone. Although control-transfected cells were unaffected by IR, the apoptosis-inducing effect of each construct was enhanced by concomitant treatment with IR (FIG. 4B). This effect was most pronounced in the case of the ORF1/2 construct, where a combination of transfection and IR reduced the number of surviving GFP-positive cells by 75% (FIG. 4B). Therefore, while ORF2 (hRpr) alone has some autonomous killing activity, this effect is enhanced both by expression of hRpr in the context of its full-length cDNA and by simultaneous stimulation with IR. The ability of ORF1 alone to produce a modest apoptotic response raises the possibility that the protein encoded by ORF1 may enhance activation of the Rpr-like pathway. As the sequence of ORF1 gives no apparent indication of its function, elucidation of this pathway will require further characterization of the ORF1 product.

Since HeLa cells are functionally deficient for p53 and do not normally die in response to IR, the synergy between hRpr and IR suggests that radiation-inducible but p53-independent pathways present in HeLa cells can cooperate with hRpr in apoptotic induction (Scheffner et al, Cell 63:1129 (1990)). Not surprisingly, endogenous hRpr protein is undetectable in untransfected HeLa cells, even following IR. In contrast, ML-1 cells (wild type p53) die by apoptosis and produce the hRpr doublet in response to IR. This induction may be mediated by a p53-binding motif noted in the intron of the hRpr gene.

Hrpr as a Potential Homolog of Fly Rpr

Based on sequence homology, Scythe interaction, radiation inducibility, and apoptotic activity, it is clear that the hRpr gene is a novel apoptotic regulator with striking similarities to fly Rpr. However, hRpr may lack one notable feature of fly Reaper—the ability to bind IAPs. In a variety of experimental approaches, it has not been possible to demonstrate a stable physical association between hRpr and IAPs. Accordingly, hRpr and Rpr are only similar, but not identical, over the region of fly Rpr which has been implicated in IAP binding (Rpr amino acids 1-15) (McCarthy and Dixit, J. Biol. Chem. 273:24009 (1998)). In preliminary studies, it appears that the Scythe-interacting and IAP-interacting regions of fly Rpr are spatially distinct and both are required for full apoptosis. These observations suggest the intriguing possibility that the two functions of the single fly Rpr have diverged evolutionarily such that, in the mammalian context, IAP inhibition and Scythe activation require two different proteins (Smac/DIABLO and hRpr, respectively). Notably, overexpression of Smac/DIABLO can enhance UV-induced cell death, but this protein itself is not a potent autonomous inducer of apoptosis, whereas transcriptional upregulation of Rpr in the fly leads to rapid cell death (Du et al, Cell 102:33 (2000); Verhagen et al, Cell 102:43 (2000)). Therefore, hRpr and Smac/DIABLO may cooperate to provide both the Scythe-dependent and IAP-inhibiting functions of fly Rpr in initiating apoptosis.

All documents cited above are hereby incorporated in their entirety by reference.

One skilled in the art will appreciate from a reading of this disclosure that various changes in form and detail can be made without departing from the true scope of the invention.

What is claimed is:

1. An isolated protein comprising the amino acid sequence shown in SEQ ID NO:2, or portion thereof of at least 10 contiguous amino acids.

2. The isolated protein according to claim 1 wherein said protein comprises the amino acid sequence shown in SEQ ID NO:2, or portion thereof of at least 30 contiguous amino acids.

3. The isolated protein according to claim 2 wherein said protein comprises the amino acid sequence shown in SEQ ID NO:2, or portion thereof of at least 50 contiguous amino acids.

4. The isolated protein according to claim 3 wherein said protein comprises the amino acid sequence shown in SEQ ID NO:2, or portion thereof of at least 75 contiguous amino acids.

5. An isolated protein comprising the amino acid sequence shown in SEQ ID NO:2 or a sequence of amino acids at least 70% homologous to the amino acid sequence shown in SEQ ID NO:2.

6. The isolated protein according to claim 5 wherein said protein comprises the amino acid sequence shown in SEQ ID NO:2 or a sequence of amino acids at least 80% homologous to the amino acid sequence shown in SEQ ID NO:2.

7. The isolated protein accoding to claim 6 wherein said protein comprises the amino acid sequence shown in SEQ ID NO:2 or a sequence of amino acids at least 90% homologous to the amino acid sequence shown in SEQ ID NO:2.

8. The isolated protein accoding to claim 7 wherein said protein comprises the amino acid sequence shown in SEQ ID NO:2 or a sequence of amino acids at least 95% homologous to the amino acid sequence shown in SEQ ID NO:2.

9. An isolated protein having the amino acid sequence shown in SEQ ID NO:2.

10. An isolated nucleic acid encoding a protein comprising the amino acid sequence shown in SEQ ID NO:2 or portion of the amino acid sequence shown in SEQ ID NO:2 of at least 10 contiguous amino acids, or complement thereof.

11. The isolated nucleic acid according to claim 10 wherein said nucleic acid encodes the protein comprising the amino acid sequence shown in SEQ ID NO:2 or portion of the amino acid sequence shown in SEQ ID NO:2 of at least 30 contiguous amino acids.

12. The isolated nucleic acid according to claim 11 wherein said nucleic acid encodes the protein comprising the amino acid sequence shown in SEQ ID NO:2 or portion of the amino acid sequence shown in SEQ ID NO:2 of at least 50 contiguous amino acids.

13. The isolated nucleic acid according to claim 12 wherein said nucleic acid encodes the protein comprising the amino acid sequence shown in SEQ ID NO:2 or portion of the amino acid sequence shown in SEQ ID NO:2 of at least 75 contiguous amino acids.

14. An isolated nucleic acid comprising the nucleotide sequence shown in SEQ ID NO:4 or a nucleotide sequence at least 70% homologous therewith, or complement thereof.

15. The isolated nucleic acid according to claim 14 wherein said nucleic acid comprises the nucleotide sequence shown in SEQ ID NO:4 or a nucleotide sequence at least 80% homologous therewith.

16. The isolated nucleic acid according to claim 15 wherein said nucleic acid comprises the nucleotide sequence shown in SEQ ID NO:4 or a nucleotide sequence at least 90% homologous therewith.

17. The isolated nucleic acid according to claim 16 wherein said nucleic acid comprises the nucleotide sequence shown in SEQ ID NO:4 or a nucleotide sequence at least 95% homologous therewith.

18. An isolated nucleic acid having the nucleotide sequence shown in SEQ ID NO:4, or complement thereof.

19. A recombinant molecule comprising a vector and the nucleic acid according to any one of claims 10, 14 and 18.

20. The recombinant molecule according to claim 19 wherein said nucleic acid is operably linked to a promoter.

21. A host cell comprising the recombinant molecule according to claim 19.

22. A method of making a protein comprising culturing the host cell according to claim 21 under conditions such that said nucleic acid is expressed and said protein is thereby produced, and isolating said protein.

23. A fusion protein comprising said protein according to any one of claims 1, 5 and 9 and a heterologous amino acid sequence.

24. An antibody specific for the protein according to any one of claims 1, 5 and 9.

25. A method of screening a test compound for its potential as a modulator of human reaper protein (hRpr) activity comprising contacting said test compound with the protein according to any one of claims 1, 5, 9 and 23 and determining whether said test compound binds said protein, wherein a test compound that binds said protein is a potential modulator of hRpr activity.

26. A potential modulator of hRpr activity identifiable by the method according to claim 25.

27. A method of treating a patient suffering from a disease or disorder that results from overexpression of hRpr comprising administering to said patient an amount of an agent that inhibits activation of apoptosis by hRpr in an amount sufficient to effect said treatment.

28. The method according to claim 27 wherein said agent is identifiable by the method according to claim 25.

29. A method treating a patient bearing a tumor comprising irradiating said tumor and administering to said patient an amount of an agent that enhances hRpr activity sufficient to effect said treatment.

30. The method according to claim 29 wherein said agent is identifiable by the method according to claim 25.

31. A kit comprising the protein according to any one of claims 1, 5, 9 and 23, or the nucleic acid according to any one of claims 10, 14 and 18 or the antibody according to claim 24, disposed within a container means.

32. The kit according to claim 31 wherein said protein, nucleic acid or antibody bears a detectable label.

33. The kit according to claim 31 wherein said protein, nucleic acid or antibody is bound to a solid support.
